(19) United States
(12) Patent Application Publication
(10) Pub. No.: US 2003/0149008 A1
(43) Pub. Date: Aug. 7, 2003

(54) Hormonal implants treatment of the breast cancer

(76) Inventor: Velayudhan Sahadevan, Beckley, WV (US)

Correspondence Address: Dr. V. Sahadevan, Dag Hammarskjold Cancer Treatment Center, 155 Dry Hill Road, Beckley, WV, 25801, US

(21) Appl. No.: 10/085,840

(22) Filed: Feb. 27, 2002

Related U.S. Application Data
(63) Continuation-in-part of application No. 10/072,416, filed on Feb. 7, 2002.

(51) Int. Cl. A61K 31/56

(52) U.S. Cl. 514/170; 424/426

(57) ABSTRACT

An improved method and products for the hormonal treatment of breast cancer by breast implants of anti-estrogens and steroid hormones in formulations as fused with a lipoid carrier or encapsulated in microcapsules or in Silastic capsules is provided. Such breast implants renders a constant slow-release of their contents to the breast tissue for extended periods by biodegradation and diffusion. It facilitates higher breast tissue concentrations of anti-estrogen and hormonal compositions. Because of their high concentration in the breast and lower systemic distribution, tumor control is much improved and the their systemic toxicity is minimized. An added beneficial effect of these breast implants on breast cancer is mediated by the inhibition of hypothalamic-pituitary LHRH, FSH and LH secretion by these composition's systemic contents. It is also an effective prophylaxis against breast cancer. Furthermore, it reduces the cost of hormonal treatment of breast cancer. Anti-estrogen hormonal implants to the breast as concomitant hormonal treatment with conventional radiation therapy also facilitates improved tumor control and cure rates of breast cancer.

### CROSS REFERENCE TO RELATED APPLICATION

This patent application is a continuation-in-part of my co-pending application No 10072416, filed on Feb. 7, 2002.

### BACKGROUND—FIELD OF THE INVENTION

This invention relates to slow release formulations of anti-estrogens and hormonal compositions for hormonal implants to the breast as an efficient but low cost treatment of the breast cancer with minimal systemic toxicity.

### BACKGROUND—DESCRIPTION OF PRIOR ART

Heretofore, hormone treatment of breast cancer is given by per oral, subcutaneous, intramuscular or intravenous injections. Because of the systemic distribution of such administrated hormones, only a very small amount of hormone will reach the target cancer cells in the breast. A great percentage of the systemically administered hormone is rapidly metabolized and eliminated from the body and hence it is wasted. Therefore patients have to take larger quantities of these hormones daily. It increases the undesirable side effects of hormone treatment making it unsafe for some patients. Daily systemic administration of the hormones also adds to the cost of these medications and hence unaffordable to some patients. Because of the very low concentration of the systemically administrated hormone reaching the cancer cells, it may not even be adequately effective in some patients.

Estrogen and or both estrogen and progesterone receptor positive tumor cells of the breast are sensitive to estrogen deprivation. Interference with estrogen signaling pathways will generate proliferative arrest of both the normal and tumor cells. Treatment with anti-estrogen tamoxifen, or raloxifene reduces the development of breast cancers (1, 2, 3, 4,5; Winer P. W. et.al, Malignant Tumors of the Breast, In Cancer, Principles and Practice of Oncology, 6^{th} edition, Vol. 1; DeVita, Jr. et al (Ed), 2001 page 1656-; (Ref. #110, 111, 112, 118,119)). Tamoxifen is also known to be a very effective drug against advanced breast cancer. The benefits of adjuvant treatment of both pre and postmenopausal estrogen receptor positive breast cancer patients with tamoxifen are proven to be substantial. Adjuvant tamoxifen treatment of patients with estrogen receptor positive tumors can reduce the annual odds of recurrence to 50-60 percent and annual odds of death to 23 to 36 per cent (6, 1; Winer P. W. et.al, Malignant Tumors of the Breast, In Cancer, Principles and Practice of Oncology, 6^{th} edition, Vol. 1; DeVita, Jr. et al (Ed), 2001 page 1690, Table 37.2-20 and Ref. #110). Similar to tamoxifen, toremifene is also an anti-estrogen used for breast cancer treatment. Like tamoxifen, toremifene also has very high tissue binding affinity (7, Erlichman C, and Loprinzi C L, Hormonal Therapies, In Cancer, Principles and Practice of Oncology, 6^{th} edition, Vol. 1; DeVita, Jr. et al (Ed), 2001 page 479-80, Ref#61). Raloxifene is another effective anti-estrogen used to treat the breast cancer (8, Erlichman C, and Loprinzi C L, Hormonal Therapies, In Cancer, Principles and Practice of Oncology, 6^{th} edition, Vol. 1; DeVita, Jr. et al (Ed), 2001 page 480). Like tamoxifen, raloxifene also prevents the breast cancer development (4,5; Winer P. W. et.al, Malignant Tumors of the Breast, In Cancer, Principles and Practice of Oncology, 6^{th} edition, Vol. 1; DeVita, Jr. et al (Ed), 2001 page 1656-; (Ref. #118,119)).

The group of patients treated concurrently with the anti-estrogen tamoxifen and external radiation had much lesser rate of breast cancer recurrence as compared with patients treated with radiation alone in the NSABP B-14 study. The rate of recurrent ipsilateral breast cancer after concurrent treatment with tamoxifen and radiation was lowered by 61 percent as compared to radiation alone (9; Winer P. W. et.al, Malignant Tumors of the Breast, In Cancer, Principles and Practice of Oncology, 6^{th} edition, Vol. 1; DeVita, Jr. et al (Ed), 2001 page 1691, Ref, #113). Concomitant anti-estrogen and lower dose conventional external beam radiation treatment is much lesser toxic and it is well tolerated. Anti-estrogen implants to the breast before and after the radiation therapy would nearly sterilize all of the focus of tumor. High efficiency anti-estrogen implant treatment with its high concentration in the breast would further improve the tumor control than those reported by Fisher et al in the NSABP B-14 study (9; Winer P. W. et.al, Malignant Tumors of the Breast, InCancer, Principles and Practice of Oncology, 6^{th} edition, Vol. 1; DeVita, Jr. et al (Ed), 2001 page 1691, Ref, #113).

The present standard dose of tamoxifen for the treatment of breast cancer or its prevention is 20 mg daily by mouth for several years. It is associated with potential risks of serious toxicities and adverse effects in terms of quality of life. It increase in the development of endometrial cancer and thromboembolism, especially among the older women (10,11; Winer P. W. et.al, Malignant Tumors of the Breast, In Cancer, Principles and Practice of Oncology, 6^{th} edition, Vol. 1; DeVita, Jr. et al (Ed), 2001 page 1657-; (Ref. #113, 114)). Like tamoxifen, treatment with raloxifene also increases the risk of thromboembolism. The risk associated with its use in developing endometrial cancer seems to have not increased by raloxifene treatment (4,5; Winer P. W. et.al, Malignant Tumors of the Breast, In Cancer, Principles and Practice of Oncology, 6^{th} edition, Vol. 1; DeVita, Jr. et al (Ed), 2001 page 1656-; (Ref. #118,119)). Among the other side effects of treatment with tamoxifen and raloxifene includes hot flashes, vaginal discharge sexual dysfunction depression and weight gain. The other commonly used hormonal therapies for breast cancer includes progestins, androgens, aminoglutethimide, LHRH analogues, glucocorticoids and oophorectomy (12, Haller D G, Fox K R, Schuchter L M, Metastatic Breast Cancer; In Breast Cancer Treatment A Comprehensive Guide to Management; Fowble B, Goodman R L, Glick J H and Rosato E F (Ed), Mosby Year Book, 1991, page 413).

The 20 mg once daily or 10 mg twice daily per oral dose of tamoxifen for three months delivers an average steady state plasma concentration of 122 ng per ml tamoxifen and 353 ng per ml of its metabolite N-desmethyl tamoxifen (13, Nolvadex, tamoxifen citrate, Zeneca Pharmaceuticals, Physicians Desk Reference, PDR 51, p 2957, 1997) The tissue bound tamoxifen distribution is much higher than that of its plasma concentration; a 10 to 60 fold higher concentration of tissue bound tamoxifen and its metabolites than its plasma concentration is observed (14, Erlichman C, and Loprinzi C L, Hormonal Therapies, In Cancer, Principles and Practice of Oncology, 6^{th} edition, Vol. 1; DeVita, Jr. et al (Ed), 2001 page 479, Ref#52). The bioavailability of orally administered tamoxifen is assumed to be about 30 percent (15, Erlichman C L, and Loprinzi C L, Hormonal Therapies, In Cancer, Principles and Practice of Oncology, 6^{th} edition, Vol. 1; DeVita, Jr. et al (Ed), 2001 page 479). Because of these very high tissue bindings of tamoxifen, by implanting relatively lower dose of tamoxifen directly to the breast, a very high steady state tamoxifen concentration is achieved from diffusion and biodegradation of the implants. Therefore the implant dose of tamoxifen need not to be as high as like its daily oral dose. Because of this 10 to 60 fold higher tissue binding of tamoxifen than its average plasma concentration and the only about 30 per cent bioavailability of the orally administered tamoxifen the tamoxifen implant dose needs only to be a smaller fraction of the oral dose. In this instance, the maintenance of a relatively high plasma concentration of tamoxifen is unnecessary. It reduces the toxicity associated with tamoxifen treatment. Furthermore, it reduces the cost for the anti-estrogen treatment of breast cancer. Because of these direct anti-estrogen implants to the breast can deliver a constant rate of anti-estrogens directly to the breast for months or years by diffusion and biodegradation, it is not dependent upon patient's compliance for its daily oral intake. The implant dose of anti-estrogen like tamoxifen is adjusted to give the same level of tissue bound anti-estrogen when it is administrated at higher dose by mouth daily. The tamoxifen implant dose for a patient is determined by comparative tamoxifen assays of needle biopsy specimens from the breast. After the oral administration of tamoxifen 10-mg by mouth twice daily for four weeks, the initial tissue bound tamoxifen is determined from needle biopsy specimens of the breast. Four weeks after interruption of the tamoxifen treatment the tamoxifen implant to the breast is made. A second comparative tissue bound tamoxifen is determined from breast needle biopsy specimens as before. If the implant dose needs to be adjusted an additional tamoxifen implants is made. Alternatively, a generally acceptable low implant dose that gives equivalent tissue bound tamoxifen as by its higher dose daily oral administration could be taken as a satisfactory implant dose. It can eliminate the routine breast needle biopsies to determine the tissue bound tamoxifen concentration.

In U.S. Pat. No. 4,321,208 (16; Sahadevan V: Preparation of directly iodinated steroid hormones and related compounds, U.S. Pat. No. 4,321,208; 1982) this inventor has described the methods for preparation of iodinated steroid hormones including the estradiol as early as in 1976. The I-125 labeled estradiol was shown to bind to estrogen antiserum and to the estrogen receptor sites. Because of the heaviness and the electronegative characteristic of iodine in the estradiol molecule, it would render cytotoxic actions to the breast cancer. Implantation iodoestradiol adsorbed sponges to rat breast tumor showed excellent tumor regression (unpublished data).

A controlled slow release implant of a depot preparation of anti-estrogen directly to the breast could achieve high concentrations of anti-estrogens to the breast and its very low concentration in the rest of the body. The ant-estrogen systemic toxicity is reduced and or eliminated by the very low levels of systemic ant-estrogen. The high levels of anti-estrogen from the implants to the breast would saturate the estrogen receptors of the breast. It would enhance the effectiveness of the anti-estrogen treatment of breast cancer.

Because of the systemic distribution of the orally administered or injected estrogens and anti-androgen compounds, only a portion of these compounds will reach the intended target site, the estrogen receptor rich breast. In addition, the methods of oral and or injectable forms of anti-estrogen administration need more disciplined compliance by the patients to take these medications daily. Furthermore, a greater percentage of such systemically distributed compounds are metabolized. Therefore, much larger doses of these compounds are needed to insure the delivery of the required dose at the target site, the breast. The commonly available pharmaceutical preparation of Depo-Provera containing medroxyprogesterone is used for contraceptive treatment. A subcutaneous implant of an oily preparation of 150 mg of medroxyprogesterone will provide 1 to 7 ng of medroxyprogesterone per ml plasma for three months (17; Pharmacia and Upjohn Company, Depo-Provera, Physicians Desk Reference, PDR, 51,1997,p2079). Implantation of steroid pellets under the skin is a well-known method of treatment with hormones.

Injections of pellets of hormones for hormone replacement treatment after oophorectomy result in large variations in serum hormone levels with high levels immediately after such injections. Hence the generally known methods of preparation of injectable slow-release depot formulations of hormones encapsulated in biodegradable polymers is made to deliver a constant dose of hormone. Similar preparations of microcapsules were described in U.S. Pat. No. 4,389,330 (18; Tice T R, and Lewis D H: Microencapsulation process, U.S. Pat. No. 4,389,330; 1983). Similar preparations are referenced and described in U.S. Pat. No. 5,340,586 (19; Pike M and Spicer D V: Methods and formulations for use in treating oophorectomized women, U.S. Pat. No. 5,340,586; 1994). Injectable encapsulated hormone preparations are made to facilitate a steady state of hormone release for periods ranging from a few days to several years and are used as subcutaneous injections for the hormone replacement treatment after oophorectomy U.S. Pat. No. 5,340,586 (19; Pike M and Spicer D V: Methods and formulations for use in treating oophorectomized women, U.S. Pat. No. 5,340,586;1994).

Several methods of preparation of pellets of compounds of steroids and other compositions are known in the art, which dates back as early as 1936 and onwards. Several of these methods are cited in the U.S. Pat. No. 4,244,949 (20; Gupta G N: Manufacture of long term contraceptive implant, U.S. Pat. No. 4,244,949; 1981) of 22 years ago, the entire disclosure of which is hereby incorporated by reference. In a preferred art for such implants preparation, the steroid is mixed with a lipoid carrier consisting of cholesterol and its organic carboxylic esters and loading and compacting this mixture into a Teflon tubing and heating the tubing at a temperature above the melting point of the steroid and lipoid under an inert gas like nitrogen, cooling the tubing and removing the pellets of fused steroid-lipoid composition. Cholesterol serves as the lipoid carrier. This formulation facilitates the constant slow release of desired dose of steroid hormone from the implanted bioabsorbable fused steroid-lipoid composition. Examples of such constant release implants of steroid hormones to provide 50 to 80 μg steroid per day in rhesus monkey is given in U.S. Pat. No. 4,244,949 (20; Gupta G N: Manufacture of long term contraceptive implant, U.S. Pat. No. 4,244,949; 1981) and which is sufficient to achieve the contraceptive effects of such formulation for one year and more in rhesus monkeys.

The U.S. Pat. No. 4,244,949 (20; Gupta G N: Manufacture of long term contraceptive implant, U.S. Pat. No. 4,244,949; 1981) uses the bioabsorbable fusion products of anti-ovulation steroid hormone and a lipoid carrier selected from the group of cholesterol for making the slow-release long acting contraceptives. Preparations of fusion products of steroid and lipoid were well known in the prior art, 23 years ago when this patent application was made. As claimed in this patent, the fused implant was made for fertility control and not as either by subcutaneous or intramuscular injections or by direct implant to the prostate for the hormonal treatment of prostate cancer.

The methods of preparations of encapsulated hormone implants described in U.S. Pat. No. 5,430,585 (21; Pike M and Spicer D V: Methods and formulations for use in treating benign gynecological disorders; U.S. Pat. Nos. 5,340,585; 1994) and 5,430,586 (19; Pike M and Spicer D V: Methods and formulations for use in treating oophorectomized women, U.S. Pat. No. 5,340,586; 1994) were also known in the prior art. Those prior art methods are discussed and referenced in these patents. U.S. Pat. No. 5,430,585 (21; Pike M and Spicer D V: Methods and formulations for use in treating benign gynecological disorders; U.S. Pat. No. 5,340,585; 1994) teaches methods and formulations of treatment of benign gynecological disorders and the U.S Pat. No. 5,340,586 (19; Pike M and Spicer D V: Methods and formulations for use in treating oophorectomized women, U.S. Pat. No. 5,340,586; 1994) teaches the methods and formulations for treatment of oophorectomized women. They do not teach the treatment of breast cancer either by subcutaneous or intramuscular injections or by direct breast implants of those encapsulated and or microspheres preparations of hormones. Furthermore, the hormonal compositions of the implant preparations of U.S. Pat. No. 5,430,585 (21; Pike M and Spicer D V: Methods and formulations for use in treating benign gynecological disorders; U.S. Pat. Nos. 5,340,585; 1994) and 5,430856 (19; Pike M and Spicer D V: Methods and formulations for use in treating oophorectomized women, U.S. Pat. No. 5,340,586; 1994) containing androgen are not suitable for the treatment of breast cancer. The steroid hormonal compositions of androgen and estrogen encapsulated in Silastic silicone tube implants were used for male contraception in U.S. Pat. No. 4,210,644 (22; Ewing L L, Desjardins C: Male contraception; U.S. Pat. No. 4,210,644; 1980). This composition is also not suitable for the treatment of prostate cancer. In the present invention described in this application, similar encapsulation methods are used to make implantable suitable hormonal compositions for the treatment of breast cancer.

Like in U.S. Pat. No. 4,210,644 (22; Ewing L L, Desjardins C: Male contraception; U.S. Pat. No. 4,210,644; 1980), the long acting synthetic progestin, the levonorgestrel encapsulated in Silastic silicone rubber tubing is used to prepare the Norplant System of Wyeth-Ayerst Laboratory's long-acting contraceptive (23;Norplant System, Wyeth Ayerst Laboratories, Physicians Desk Reference, PDR, 51, 1997, p2868). Implantation of this long acting encapsulated contraceptive levonorgestrel protects from fertility up to 5 years. These implants are usually implanted subcutaneousely to the upper arm. After 5 years, the inert and empty Silastic capsule is removed from the implant site. This formulation is also not for the treatment of breast cancer.

### OBJECTS AND ADVANTAGES

It is therefore, an object of this invention to provide a less or no toxic improved method of hormonal treatment of breast cancer and said hormonal treatment comprising of implanting anti-estrogens in one or more slow release formulations and permitting said drugs to be continuously released at near constant rate directly to the breast for longer periods and maintaining said formulation's serum level low as to minimize or to eliminate systemic toxicity.

It is another object of the invention to provide slow-release biodegradable seeds or microcapsules or Silastic capsules containing anti-estrogenic compositions for breast implant for the hormonal treatment of breast cancer.

Another object of the invention is to provide slow-release anti-estrogen implant products for treating breast cancer with less toxicity and cost as an alternative to daily oral administration of high dose anti-estrogens and said implants consisting of implanting biodegradable seeds or microcapsules or Silastic capsules containing said anti-estrogen formulations to deliver high concentrations of said hormonal formulations to the breast for longer periods.

Still another object of this invention is to provide high concentrations of anti-estrogen formulations in the breast by said formulation's direct implant in the breast to obviates the necessity of daily systemic administration of said compositions in higher doses for the treatment of breast cancer.

It is a further object of this invention to maintain high concentrations of anti-estrogen formulations in the breast by implanting slow-release biodegradable seeds or microcapsules or Silastic capsules containing anti-estrogen compositions for breast implant to maintain such composition's systemic concentration low by dilution of said released formulations through circulation and thereby eliminate or minimize the systemic toxicity associated with such formulations.

It is a further object of this invention to make implants of iodo-estradiol as slow-release biodegradable seeds or microcapsules or Silastic capsules containing said compositions for breast implants to deliver high concentrations of said formulations to the breast.

It is still another object of this invention to make implants of natural corticosteroids and their synthetic derivatives alone or in combination with anti-estrogens as slow-release biodegradable seeds or microcapsules or Silastic capsules containing said compositions for breast implants to deliver high concentrations of said formulations to the breast for the treatment of hormone refractory breast cancer with lesser toxicity than by said formulation's higher dose systemic administration by oral, subcutaneous or intramuscular routes.

Still it is another object of this invention to make steroid hormonal implants alone or in combination with anti-estrogen compositions containing in slow-release biodegradable seeds or microcapsules or in Silastic capsules for breast implants to deliver high concentrations of said formulations to the breast and to suppress the hypothalamic-pituitary LHRH, LH and FSH secretion by their lower systemic concentrations for the treatment of hormone dependent and hormone refractory breast cancer with lesser toxicity than by said formulation's higher dose systemic administration by oral, subcutaneous or intramuscular routes.

It is still a further object of this invention to make implants of natural progesterone and its synthetic derivatives alone or in combination with anti-estrogen as slow-release biodegradable seeds or microcapsules or Silastic capsules containing said compositions for breast implants to deliver high concentrations of said formulations to the breast with lesser toxicity than by said formulation's higher dose systemic administration by oral, subcutaneous or intramuscular routes.

It is a further object of this invention to make prostate implants of anti-estrogen compounds as fused with a lipoid carrier, or as injectable microcapsules or encapsulated in Silastic capsules to achieve slow release of said compounds by diffusion and biodegradation of the carrier or by diffusion alone and the slowly released anti-estrogen to bind and to saturate the estrogen receptor sites in the breast competitively with estrogens to block the growth and proliferation of the breast cancer with lesser systemic toxicity than by said compound's daily high dose systemic administration.

It is still another object of this invention to make implants of anti-estrogen compositions as slow-release biodegradable seeds or microcapsules or Silastic capsules containing said compositions for implants to gross metastatic breast cancer to deliver high concentrations of said formulations to said metastasis as an efficient method of treatment of hormone dependent metastasis of the breast cancer with lesser toxicity than by said formulation's higher dose systemic administration by oral, subcutaneous or intramuscular routes.

It is a further object of this invention to reduce the cost of present hormonal treatment of breast cancer substantially by direct breast implants of long acting anti-estrogen compounds and to increase the efficiency of such treatments but with lesser toxicity than by said compounds daily systemic administration.

A further object of this invention is to minimize or to eliminate side effects such as thromboembolic events associated with treatments of breast cancer with anti-estrogens by minimizing its systemic concentration and maximizing its breast contents by implanting said implants directly to the breast and allowing slow release of such compositions from the implants to the prostate by diffusion and biodegradation.

A further object of this invention is to minimize or to eliminate the side effects of anti-estrogen treatments of breast cancer such as hot flashes, weight gain, vaginal bleeding and discharge, endometrial cancer by maintaining its low systemic dose while maintaining its high breast contents by release of the contents of said implants directly to the breast by diffusion and biodegradation.

It is another object of this invention to make implants of cytotoxic drugs alone or in combination with anti-estrogen compositions as slow-release longer lasting biodegradable seeds or microcapsules or Silastic capsules containing said compositions for breast implants to deliver high concentrations of said formulations to the breast for an extended period as part of concomitant radiation and hormonal treatment with lesser toxicity than by said formulation's higher dose systemic administration by oral, subcutaneous or intramuscular routes.

It is still another object of this invention to make implants of anti-estrogen compositions as slow-release longer lasting biodegradable seeds or microcapsules or Silastic capsules containing said compositions for breast implants and maintaining of said drug compositions for extended periods by diffusion and biodegradation from said breast implants at an amount effective to suppress focal tumor development as prophylaxis with minimum or no systemic toxicity.

Still further objects and advantages will become apparent from a consideration of the ensuing descriptions.

### SUMMARY

Estrogen and or both estrogen and progesterone receptor positive tumor cells of the breast are sensitive to estrogen deprivation. Anti-estrogen compounds like tamoxifen or raloxifene competitively binds to estrogen receptor protein with estrogen. Interference with estrogen signaling pathways by anti-estrogens will generate proliferative arrest of both the normal and tumor cells. Treatment with anti-estrogen tamoxifen or raloxifene reduces the development of breast cancers. Adjuvant treatment of patients with estrogen receptor positive tumors with tamoxifen reduces the annual odds of recurrent breast cancer to 50-60 percent and annual odds of death from breast cancer to 23 to 36 per cent. Tamoxifen is also a very effective anti-estrogen drug against advanced breast cancer.

The present standard dose of anti-estrogen like the tamoxifen for the treatment of breast cancer or its prevention is 20 mg daily by mouth. Duration of such anti-estrogen treatment is for several years. It is associated with potential risks of serious toxicities and adverse effects in terms of quality of life. It increase in the development of endometrial cancer and thromboembolism, especially among the older women

Because of the systemic distribution of the orally administered or injected anti-estrogen compounds, only a portion of these compounds will reach the intended target site, the breast. A greater percentage of such systemically distributed compounds are metabolized. Therefore, much larger doses of these compounds are taken daily or very frequently to insure the delivery of the required dose to the breast, which increases its systemic toxicity and the cost. This invention is aimed to improve the effectiveness of anti-estrogen treatment of breast cancer and to reduce such treatment associated toxicity and cost by anti-estrogen implants to breast.

Breast implants of androgen suppressive drugs formulated as fused with a lipoid carrier or encapsulated in microcapsules or in Silastic capsules render a constant slow-release of their contents to the breast for extended periods by biodegradation and diffusion. They facilitate higher breast and lower systemic concentrations of anti-estrogen compositions. Because of their high concentrations in the breast, tumor control is much more improved. Because of the anti-estrogen's lower systemic concentrations, their toxicity is minimized or eliminated.

Interference with estrogen signaling pathways by anti-estrogens will generate proliferative arrest of tumor cells and would enhance the effectiveness radiation therapy after the removal of localized breast cancer. Like the improved effective tumor control by concomitant radiation and hormonal treatment of prostate cancer, after surgical removal of localized breast cancer, the concomitant anti-estrogen and conventional radiation therapy will improve the recurrence of breast cancer. It would also facilitate a lower dose radiation to the breast after lumpectomy. Lower dose conventional radiation combined with anti-estrogen treatment is a much less toxic treatment than the higher dose radiation alone.

### DETAILED DESCRIPTION OF THE INVENTION

Pursuant to the present invention, the method of breast cancer treatment with anti-estrogen is improved by direct breast implants of such composition's depot formulations. The therapeutic effectiveness of such depot formulation is significantly improved by maintaining such formulation's higher concentration in the breast. Because of its systemic dilution, its serum concentration is much low. This low-level of systemic concentration of the anti-estrogen compounds diminishes and or eliminates many of the side effects associated with their daily oral administration. The direct breast implants of anti-estrogen compositions facilitate complete saturation of its binding sites in the breast.

A number of methods for preparing formulations of slow-release long-acting compositions of hormones are described in many of the prior arts. Such methods of preparations of slow-release long-acting hormonal compositions include the bioabsorbable fusion products of steroid and a lipoid carrier as described in U.S. Pat. No. 4,244,949 (20; Gupta G N: Manufacture of long term contraceptive implant, U.S. Pat. No. 4,244,949; 1981). Preparations of microcapsules laden with an active ingredient are described in U.S. Pat. No. 4,389,330 (18; Tice T R, and Lewis D H: Microencapsulation process, U.S. Pat. No. 4,389,330; 1983) in 1983. Similar biodegradable injectable microcapsules made of hormones and polymers such as polyortho-ester or polyacetal were used in U.S. Pat. No. 5,430,585 (21; Pike M and Spicer D V: Methods and formulations for use in treating oophorectomized women, and in 19; U.S. Pat. No. 5,340,586; 1994). Hormonal compositions as slow-release capsules made of Silastic, Dow Corning's No 602-305 medical grade polydimethylsiloxane, an inert non-reactive tube forming polymer was used to encapsulate the hormone compositions in U.S. Pat. No. 4,210,644 (22; Ewing L L, Desjardins C: Male contraception; U.S. Pat. No. 4,210,644; 1980). As in U.S. Pat. No. 4,210,644, Silastic silicone rubber tubing is used for the preparation of levonorgestrel implant, Norplant System of Wyeth -Ayerst Laboratories as a long-acting contraceptive (23; Norplant System, Wyeth Ayerst Laboratories, Physicians Desk Reference, PDR, 51, 1997, p2868). In this invention, similar prior arts methods are adapted to prepare suitable implants of anti-estrogen formulations for the implant treatment of breast cancer.

### PREFERRED EMBODIMENT—DESCRIPTION

### Preparation of Biodegradable Hormonal Compositions Fused With a Lipoid Carrier for Breast Implants

As a preferred method of fused implant preparation for breast implants for hormonal treatment of breast cancer, the methods described in U.S. Pat. No. 4,244,949 (20; Gupta G N: Manufacture of long term contraceptive implant, U.S. Pat. No. 4,244,949; 1981) more than 21 years ago is adapted. The entire disclosure of which is hereby incorporated by reference.

1. Preparation of Biodegradable Fused Breast Implants of Tamoxifen and Cholesterol Formulation

In accordance with one preferred embodiment for one fused implant preparation of tamoxifen and cholesterol for prostatic implant, tamoxifen is purified by dissolving it in methanol, filtering through analytical grade filter paper and crystallizing it by slow addition of small amount of distilled water and allowing it to continue to crystallize slowly in a refrigerator for about 12 hours. Filtering it again through analytical grade filter paper and vacuum drying at 60° C. to a constant weight for two or more hours and storing the crystallized form of under nitrogen at 25° C. until it is used for fused single breast implant preparation. Thirty mg of purified tamoxifen and 7.5 mg of cholesterol is made to a powder form by thorough mixing under nitrogen. This mixture is then transferred into a 10 cm long, 2.4 to 2.8 mm diameter Teflon tubing and compacted with stainless steel probes from both open ends of the Teflon tubing under nitrogen. The portion of the Teflon tubing containing the tamoxifen and cholesterol mixture is heated over their melting points for 45 seconds over an aluminum block. The molten mixture is consolidated as one fused mass by pressing it with the stainless steel probes. After cooling, the probes are removed. The fused tamoxifen and cholesterol breast implant preparation is removed from the Teflon tubing by splitting the tube walls with a blade. As described earlier, the implant dose of tamoxifen is adjusted to give the same amount of breast tissue bound tamoxifen as 4 weeks after high dose tamoxifen treatment by mouth.

2. Preparation of Biodegradable Fused Breast Implants of Raloxifene and Cholesterol Formulation

In accordance with one preferred embodiment for one fused implant preparation of raloxifene and cholesterol for the breast implants, the methods similar to that described for the preparation of tamoxifen fused with cholesterol is used but substituting the tamoxifen with raloxifene and adjusting the amount of raloxifene and cholesterol used for such preparation.

3. Preparation of Iodinated Estradiol (Iodo-Estradiol)

Iodinated estradiol is prepared as per the methods described by this inventor in his U.S. Pat. No. 4,321,208 in 1982 with minor modifications (16; Sahadevan V: Preparation of directly iodinated steroid hormones and related compounds, U.S. Pat. No. 4,321,208; 1982). In brief, non-radioactive iodoestradiol is prepared by dissolving estradiol in methanol and allowing it to react with iodine. In a preferred embodiment, sodium or potassium iodide is dissolved in water. Hydrogen peroxide or chloramine-T dissolved in small amount of water is added to free the elemental iodine from its sodium or potassium salts. Iodine reactions with estrogen molecules take place spontaneously and form the iodoestradiol. The iodinated estradiol is precipitated with water and it is separated from the reaction mixture by centrifugation.

In a preferred embodiment 8 gr. Estradiol 17-β is dissolved in 100-ml methanol and filtered through analytical filter paper. Separately, 1-gr. sodium iodide and 100 μg chloramine-T is dissolved in 5-ml water and this is added to the estradiol dissolved in methanol. The iodine labeling to estradiol takes place spontaneously. After this reaction mixture is allowed to stand for about an hour, at room temperature, about 100 ml distilled water is added slowly to precipitate the iodoestradiol. The reaction mixture is centrifuged and the sediment iodoestradiol is washed repeatedly with water to remove any residual of iodine and chloramine-T. The sediment of iodoestradiol is vacuum dried at 60° C. for two or more hours to a constant weight and it is stored under nitrogen at 25° C. until it is used.

As shown by this inventor (16; Sahadevan V: Preparation of directly iodinated steroid hormones and related compounds, U.S. Pat. No. 4,321,208; 1982), such iodinated estradiol binds to both the estrogen receptor sites and to estrogen antiserum indicating its similarity with the naturally occurring estradiol 17β.

4. Preparation of Biodegradable Fused Breast Implants of Iodoestradiol and Cholesterol Formulation.

In accordance with one preferred embodiment for one fused implant preparation of iodoestradiol and cholesterol for breast implant, the methods similar to that described for the preparation of tamoxifen fused with cholesterol is used but substituting the tamoxifen with iodo-estradiol and adjusting the amount of iodo-estradiol and cholesterol used for such preparation.

5. Preparation of Biodegradable Fused Breast Implants of Toremifene and Cholesterol Formulation

In accordance with one preferred embodiment for one fused implant preparation of toremifene and cholesterol for breast implant, the methods similar to that described for the preparation of tamoxifen fused with cholesterol is used but substituting the tamoxifen with toremifene and adjusting the amount of toremifene and cholesterol used for such preparation.

6. Preparation of Biodegradable Fused Breast Implants of Progesterone and Cholesterol Formulation

In accordance with one preferred embodiment for one fused implant preparation of progesterone and cholesterol for breast implant, the methods similar to that described for the preparation of tamoxifen fused with cholesterol is used but substituting the tamoxifen with progesterone and adjusting the amount of progesterone and cholesterol used for such preparation.

7. Preparation of Biodegradable Fused Breast Implants of Androgen Fluoxymesterone and Cholesterol Formulation

In accordance with one preferred embodiment for one fused implant preparation of fluoxymesterone and cholesterol for breast implant, the methods similar to that described for the preparation of tamoxifen fused with cholesterol is used but substituting the tamoxifen with fluoxymesterone and adjusting the amount of fluoxymesterone and cholesterol used for such preparation.

8. Preparation of Biodegradable Fused Breast Implants of Prednisolone and Cholesterol Formulation

In accordance with one preferred embodiment for one fused implant preparation of prednisolone and cholesterol for breast implant, the methods similar to that described for the preparation of tamoxifen fused with cholesterol is used but substituting the tamoxifen with prednisolone and adjusting the amount of prednisolone and cholesterol used for such preparation.

9. Preparation of Biodegradable Fused Breast Implants of Anti-Estrogens combined with Progestins and Cholesterol Formulation

In accordance with one preferred embodiment for one fused implant preparation of ant-estrogens combined with progestins and cholesterol for breast implant, the methods similar to that described for the preparation of tamoxifen fused with cholesterol is adapted. An anti-estrogen is selected from the group of tamoxifen, raloxifene or toremifene. A progestin is selected from the group of megestrol acetate, medroxyprogesterone, norethindrone acetate or norgestrel. The amount of the selected anti-estrogen, progestin and cholesterol is adjusted for the preparation of the said fused combination hormonal breast implant of anti-estrogen combined with a progestin and cholesterol.

10. Preparation of Biodegradable Fused Breast Implants of Anti-Estrogens Combined With Prednisolone and Cholesterol Formulation

In accordance with one preferred embodiment for one fused implant preparation of anti-estrogens combined with progestins and cholesterol for breast implant, the methods similar to that described for the preparation of tamoxifen fused with cholesterol is adapted. An anti-estrogen is selected from the group of tamoxifen, raloxifene or toremifene. The amount of the selected anti-estrogen, prednisolone and cholesterol is adjusted for the preparation of the said fused combination hormonal breast implant of anti-estrogen combined with prednisolone and cholesterol.

11. Preparation of Biodegradable Fused Breast Implants of Anti-Estrogens combined with an Androgen and Cholesterol Formulation

In accordance with one preferred embodiment for one fused implant preparation of anti-estrogens combined with progestins and cholesterol for breast implant, the methods similar to that described for the preparation of tamoxifen fused with cholesterol is adapted. An anti-estrogen is selected from the group of tamoxifen, raloxifene or toremifene. The amount of the selected anti-estrogen, and the androgen like the fluoxymesterone and cholesterol is adjusted for the preparation of the said fused combination hormonal breast implant of anti-estrogen combined with an androgen and cholesterol.

12. Preparation of Biodegradable Fused Breast Implants of Anti-Estrogens, Progestins, Prednisolone and Cholesterol Formulation

In accordance with one preferred embodiment for one fused implant preparation of anti-estrogens combined with progestins, prednisolone and cholesterol for breast implant, the methods similar to that described for the preparation of tamoxifen fused with cholesterol is adapted. An anti-estrogen is selected from the group of tamoxifen, raloxifene or toremifene. A progestin is selected from the group of megestrol acetate, medroxyprogesterone, norethindrone acetate or norgestrel. The amount of the selected anti-estrogen, progestin, prednisolone and cholesterol is adjusted for the preparation of the said fused combination hormonal breast implant of anti-estrogen combined with a progestin, prednisolone and cholesterol.

### Preparation of Slow-Release Hormonal Compositions in Silastic Capsules for Breast Implants

As a preferred method of slow-release Hormonal Compositions in Silastic Capsules for prostatic implants for hormonal treatment of breast cancer, the methods described in U.S. Pat. No. 4,210,644 (22; Ewing L L, Desjardins C: Male contraception; U.S. Pat. No. 4,210,644; 1980) more than 21 years ago is adapted. The entire disclosure of which is hereby incorporated by reference. Similar encapsulated levonorgestrel implant, Norplant System of Wyeth-Ayerst Laboratories is used as a long-acting contraceptive (23; Norplant System, Wyeth Ayerst Laboratories, Physicians Desk Reference, PDR, 51, 1997, p2868).

1. Preparation of Silastic Slow-Release Capsules Containing Tamoxifen for Breast Implant

In accordance with one preferred embodiment for preparation of Silastic slow release capsules containing Tamoxifen for prostatic implant, the following method is adapted. The Dow Corning Silastic, dimethylsyloxane/methylvinyalsiloxane copolymer, tubing of 0.2-mm wall thickness and 2.4 to 3.18 mm-outer diameters and of 3.5 cm in length is cut. One end is closed with Silastic adhesive (polydimethylsiloxane). Tamoxifen is filled into the cut tube through the open end at a dose of 30 mg. After the filling with DES, the open end of the tube is also closed with Silastic adhesive.

2. Preparation of Silastic Slow-Release Capsules Containing Raloxifene for Breast Implant

In accordance with one preferred embodiment for preparation of Silastic slow release capsules containing raloxifene for the breast implants, the methods similar to that described for the preparation of Silastic slow release capsules containing tamoxifen is used but substituting the tamoxifen with raloxifene and adjusting the amount of raloxifene used for such preparation.

3. Preparation of Silastic Slow-Release Capsules Containing Iodo-Estradiol for Breast Implant

In accordance with one preferred embodiment for preparation of Silastic slow release capsules containing raloxifene for the breast implants, the methods similar to that described for the preparation of Silastic slow release capsules containing tamoxifen is used but substituting the tamoxifen with iodo-estradiol and adjusting the amount of iodo-estradiol used for such preparation.

4. Preparation of Silastic Slow-Release Capsules Containing Toremifene for Breast Implant

In accordance with one preferred embodiment for preparation of Silastic slow release capsules containing toremifene for the breast implants, the methods similar to that described for the preparation of Silastic slow release capsules containing tamoxifen is used but substituting the tamoxifen with toremifene and adjusting the amount of toremifene used for such preparation.

5. Preparation of Silastic Slow-Release Capsules Containing Progesterone for Breast Implant

In accordance with one preferred embodiment for preparation of Silastic slow release capsules containing progesterone for the breast implants, the methods similar to that described for the preparation of Silastic slow release capsules containing tamoxifen is used but substituting the tamoxifen with progesterone and adjusting the amount of progesterone used for such preparation.

6. Preparation of Silastic Slow-Release Capsules Containing Androgen Fluoxymesterone for Breast Implant

In accordance with one preferred embodiment for preparation of Silastic slow release capsules containing fluoxymesterone for the breast implants, the methods similar to that described for the preparation of Silastic slow release capsules containing tamoxifen is used but substituting the tamoxifen with fluoxymesterone and adjusting the amount of fluoxymesterone used for such preparation.

7. Preparation of Silastic Slow-Release Capsules Containing Prednisolone for Breast Implant

In accordance with one preferred embodiment for preparation of Silastic slow release capsules containing prednisolone for the breast implants, the methods similar to that described for the preparation of Silastic slow release capsules containing tamoxifen is used but substituting the tamoxifen with prednisolone and adjusting the amount of prednisolone used for such preparation.

8. Preparation of Silastic Slow-Release Capsules Containing Anti-Estrogens and Progestins for Breast Implant

In accordance with one preferred embodiment for preparation of Silastic slow release capsules containing anti-estrogens combined with progestins for breast implant, the methods similar to that described for the preparation of Silastic slow release capsules containing tamoxifen is adapted An anti-estrogen is selected from the group of tamoxifen, raloxifene or toremifene. A progestin is selected from the group of megestrol acetate, medroxyprogesterone, norethindrone acetate or norgestrel. The amount of the selected anti-estrogen and the progestin is adjusted for the preparation of the said Silastic slow release capsules containing the combination hormonal breast implant of anti-estrogen and a progestin.

9. Preparation of Silastic Slow-Release Capsules Containing Anti-Estrogens and Prednisolone for Breast Implant

In accordance with one preferred embodiment for preparation of Silastic slow release capsules containing anti-estrogens combined with prednisolone for breast implant, the methods similar to that described for the preparation of Silastic slow release capsules containing tamoxifen is adapted. An anti-estrogen is selected from the group of tamoxifen, raloxifene or toremifene. The amount of the selected anti-estrogen and the prednisolone is adjusted for the preparation of the said Silastic slow release capsules containing the combination hormonal breast implant of an anti-estrogen and prednisolone.

10. Preparation of Silastic Slow-Release Capsules Containing Anti-Estrogens and Fluoxymesterone for Breast Implant

In accordance with one preferred embodiment for preparation of Silastic slow release capsules containing anti-estrogens combined with fluoxymesterone for breast implant, the methods similar to that described for the preparation of Silastic slow release capsules containing tamoxifen is adapted. An anti-estrogen is selected from the group of tamoxifen, raloxifene or toremifene. The amount of the selected anti-estrogen and the fluoxymesterone is adjusted for the preparation of the said Silastic slow release capsules containing the combination hormonal breast implant of an anti-estrogen and fluoxymesterone.

11. Preparation of Silastic Slow-Release Capsules Containing Anti-Estrogens, Progestins and Prednisolone for Breast Implant

In accordance with one preferred embodiment for preparation of anti-estrogens combined with progestins and prednisolone for breast implant, the methods similar to that described for the preparation of Silastic slow-release capsules containing tamoxifen is adapted. An anti-estrogen is selected from the group of tamoxifen, raloxifene or toremifene. A progestin is selected from the group of megestrol acetate, medroxyprogesterone, norethindrone acetate or norgestrel. The amount of the selected anti-estrogen, progestin, and prednisolone is adjusted for the preparation of the said combination hormonal breast implant of anti-estrogen combined with a progestin and prednisolone.

### Preparation of Slow-Release Hormonal Compositions in Microcapsules for Breast Implants

As a preferred method of slow-release hormonal compositions in microcapsules for the treatment of breast cancer as breast implants, the methods described in U.S. Pat. No. 4,389,33018 (18; Tice T R, and Lewis D H: Microencapsulation process, U.S. Pat. No. 4,389,330; 1983) more than 18 years ago is adapted. The entire disclosure of which is hereby incorporated by reference. Similar methods of preparations of biodegradable microencapsulated steroid hormones are used in U.S. Pat. No. 5,340,585 (21; Pike M and Spicer D V: Methods and formulations for use in treating benign gynecological disorders; U.S. Pat. No. 5,340,585; 1994) for the treatment of benign gynecological disorders and in U.S. Pat. No. 5,340,586 (19; Pike M and Spicer D V: Methods and formulations for use in treating oophorectomized women, U.S. Pat. No. 5,340,586; 1994) for use of treating oophorectomized women. They are also hereby incorporated by reference. Similarly, any of the many previously known prior art methods for the preparation of microencapsulated compositions could also be used for the preparation of microencapsulated steroid hormones and their synthetic derivatives as breast implants for the treatment and prevention of breast cancer of this invention.

1. Preparation of Slow-Release Biodegradable Microcapsules Containing Tamoxifen for Breast Implant

In accordance with one preferred embodiment for preparation of slow-release biodegradable microcapsules containing tamoxifen for breast implant, the following method is adapted. 3 g of tamoxifen and 3 g of poly(dl-lactide-coglycolide) are dissolved in 18 g of methylene chloride and dispersed as stable emulsions of microdroplets in 58 g of wt % of aqueous poly(vinyl alcohol). Afterwards, 60% of the solvent methylene chloride was removed by evaporation. The tamoxifen containing microcapsules are removed by centrifugation. The sediment of microencapsulated tamoxifen is then resuspended in deionized water and filtered through a fine fritted-glass funnel by slow suction while continuously adding more deionized water to remove the residual methylene chloride. This filtered microencapsulated tamoxifen is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

2. Preparation of Slow-Release Biodegradable Microcapsules Containing Raloxifene for Breast Implant

In accordance with one preferred embodiment for preparation of slow release biodegradable microcapsules containing raloxifene for the breast implants, the methods similar to that described for the preparation of slow release biodegradable microcapsules containing tamoxifen is used but substituting the tamoxifen with raloxifene and adjusting the amount of raloxifene used for such preparation.

3. Preparation of Slow-Release Biodegradable Microcapsules Containing Iodo-Estradiol for Breast Implant

In accordance with one preferred embodiment for preparation of slow release biodegradable microcapsules containing raloxifene for the breast implants, the methods similar to that described for the preparation of slow release biodegradable microcapsules containing tamoxifen is used but substituting the tamoxifen with iodo-estradiol and adjusting the amount of iodo-estradiol used for such preparation.

4. Preparation of Slow-Release Biodegradable Microcapsules Containing Toremifene for Breast Implant

In accordance with one preferred embodiment for preparation of slow release biodegradable microcapsules containing toremifene for the breast implants, the methods similar to that described for the preparation of slow release biodegradable microcapsules containing tamoxifen is used but substituting the tamoxifen with toremifene and adjusting the amount of toremifene used for such preparation.

5. Preparation of Slow-Release Microcapsules Containing Progesterone for Breast Implant

In accordance with one preferred embodiment for preparation of slow release biodegradable microcapsules containing progesterone for the breast implants, the methods similar to that described for the preparation of slow release biodegradable microcapsules containing tamoxifen is used but substituting the tamoxifen with progesterone and adjusting the amount of progesterone used for such preparation.

6. Preparation of Slow-Release Biodegradable Microcapsules Containing Androgen Fluoxymesterone for Breast Implant

In accordance with one preferred embodiment for preparation of Silastic slow release biodegradable microcapsules containing fluoxymesterone for the breast implants, the methods similar to that described for the preparation of slow release biodegradable microcapsules containing tamoxifen is used but substituting the tamoxifen with fluoxymesterone and adjusting the amount of fluoxymesterone used for such preparation.

7. Preparation of Silastic Slow-Release Biodegradable Microcapsules Containing Prednisolone for Breast Implant

In accordance with one preferred embodiment for preparation of slow release biodegradable microcapsules containing prednisolone for the breast implants, the methods similar to that described for the preparation of slow release biodegradable microcapsules containing tamoxifen is used but substituting the tamoxifen with prednisolone and adjusting the amount of prednisolone used for such preparation.

8. Preparation of Silastic Slow-Release Capsules Containing Anti-Estrogens and Progestins for Breast Implant

In accordance with one preferred embodiment for preparation of Silastic slow release biodegradable microcapsules containing anti-estrogens combined with progestins for breast implant, the methods similar to that described for the preparation of slow release biodegradable microcapsules containing tamoxifen is adapted. An anti-estrogen is selected from the group of tamoxifen, raloxifene or toremifene. A progestin is selected from the group of megestrol acetate, medroxyprogesterone, norethindrone acetate or norgestrel. The amount of the selected anti-estrogen and the progestin is adjusted for the preparation of the said biodegradable slow release microcapsules containing the combination hormonal breast implant of anti-estrogen and a progestin.

9. Preparation of Slow-Release Biodegradable Microcapsules Containing Anti-Estrogens and Prednisolone for Breast Implant

In accordance with one preferred embodiment for preparation of slow release biodegradable microcapsules containing anti-estrogens combined with prednisolone for breast implant, the methods similar to that described for the preparation of slow release biodegradable microcapsules containing tamoxifen is adapted. An anti-estrogen is selected from the group of tamoxifen, raloxifene or toremifene. The amount of the selected anti-estrogen and the prednisolone is adjusted for the preparation of the said slow release biodegradable microcapsules containing the combination hormonal breast implant of an anti-estrogen and prednisolone.

10. Preparation of Slow-Release Biodegradable Microcapsules Containing Anti-Estrogens and Fluoxymesterone for Breast Implant

In accordance with one preferred embodiment for preparation of Silastic slow release biodegradable microcapsules containing anti-estrogens combined with fluoxymesterone for breast implant, the methods similar to that described for the preparation of slow release biodegradable microcapsules containing tamoxifen is adapted. An anti-estrogen is selected from the group of tamoxifen, raloxifene or toremifene. The amount of the selected anti-estrogen and the fluoxymesterone is adjusted for the preparation of the said slow release biodegradable microcapsules containing the combination hormonal breast implant of an anti-estrogen and fluoxymesterone.

11. Preparation of Slow-Release Biodegradable Microcapsules Containing Anti-Estrogens, Progestins and Prednisolone for Breast Implant

In accordance with one preferred embodiment for preparation of anti-estrogens combined with progestins and prednisolone for breast implant, the methods similar to that described for the preparation of slow-release biodegradable microcapsules containing tamoxifen is adapted. An anti-estrogen is selected from the group of tamoxifen, raloxifene or toremifene. A progestin is selected from the group of megestrol acetate, medroxyprogesterone, norethindrone acetate or norgestrel. The amount of the selected anti-estrogen, progestin, and prednisolone is adjusted for the preparation of the said combination hormonal breast implant of anti-estrogen combined with a progestin and prednisolone.

### PREFERRED EMBODIMENT—OPERATION

### Oral Pre-Implant Treatment With Tamoxifen

To determine the level of tissue bound tamoxifen after the standard dose of 10 mg twice daily by mouth for four weeks, the initial treatment with tamoxifen will be started at this standard dose for four weeks. If an anti-estrogen other than tamoxifen or an anti-estrogen in combination with other hormonal implant is planned, then the pre-implant treatment will consist of such oral preparations in its standard dose. Afterwards, a needle biopsy from the breast is taken and the concentration of the tissue bound anti-estrogen and other hormone is determined. It is followed by no treatment with anti-estrogen and other hormones for four weeks to allow the clearance of the tissue bound anti-estrogen and other hormones.

### Breast Implants of the Anti-Estrogen Formulations

A formulation of slow-release anti-estrogen from any one of the preparations described before is implanted to the breast subcutaneousely. Four weeks afterwards, repeat needle biopsy specimen from the breast is taken for the determination of the tissue bound anti-estrogen. If implants of anti-estrogen in combination with other hormones are used, its tissue levels are also determined. It is compared with the tissue bound anti-estrogen or that of in combination with other hormones four weeks earlier. If the pre-treatment oral standard dose treatment shows a higher level of tissue bound anti-estrogen, additional implants are made for dose adjustments. Similar needle biopsies from the breast tissue are taken periodically to determine the satisfactory levels of tissue bound anti-estrogen and other hormones that were included in the implant.

### Concomitant Hormonal Implant Treatment of the Breast With Radiation Therapy

The concomitant hormonal treatment with radiation is known to improve the treatment outcome of prostate cancer. Treatment with tamoxifen while on external beam radiation is shown to have improved tumor control. The slow constant rate hormonal release from the hormonal implants to the breast combined with radiation is an effective means to control the breast cancer and its cure. Furthermore, it would facilitate cure and control of breast cancer with lesser and better tolerated dose of radiation.

The hormonal implants to the breast is done either before or concomitantly with the interstitial radioactive seed implants to the breast. An added advantage of such combined hormonal implant and external radiation therapy is that it also effectively controls regional lymph node metastasis since these hormonal compositions from the biodegrading implants will be carried to the regional lymph nodes by the macrophages.

### Hormonal Breast Implants for Prophylaxis Against Breast Cancer

Treatment with tamoxifen is a very effective prophylaxis against breast cancer. Adjuvant tamoxifen treatment of patients with estrogen receptor positive tumors can reduce the annual odds of recurrence to 50-60 percent and annual odds of death to 23 to 36 per cent. The slow-release anti-estrogen hormonal implants to the breast that will maintain the breast tissue saturated with the anti-estrogen for longer periods. The breast tissue has high affinity binding for anti-estrogens. The breast tissue bound tamoxifen is about 10 to 60 times higher than its plasma concentration. Therefore, the slow-release anti-estrogen hormonal implant to the breast is an effective hormonal prophylactic treatment without much systemic toxicity.

### CONCLUSIONS, RAMIFICATIONS, AND SCOPE

Although the description above contains much specificity, these should not be construed as limiting the scope of the invention but as merely providing illustrations of some of the presently preferred embodiments of this invention. Various other embodiments and ramifications are possible within it's scope. For example, instead of the direct breast implants of anti-estrogen and related hormonal formulations that are beneficial for the treatment of breast cancer, they may be also be implanted as subcutaneous or intramuscular implants for the treatment of breast cancer. They may also be implanted directly to a metastatic site.

Thus the scope of the invention should be determined by the appended claims and their legal equivalents, rather than by the examples given.

What is claimed is:

1. An improved method of hormonal treatment of breast cancer and said hormonal treatment comprising of breast implants of anti-estrogens and steroid hormones, or its synthetic derivatives in one or more slow release formulations and permitting said drugs to be continuously released at near constant rate directly to the breast for longer periods with minimal or no systemic toxicity.

2. A method according to claim 1 further comprising release of said anti-estrogen and hormonal compositions to the breast for extended periods by diffusion and biodegradation from said breast implants in sufficient amounts to saturate the binding sites for said drug compositions in the breast and to exert their maximum tumor control activity.

3. A method of claim 1 wherein said implants comprising of hormonally effective compositions selected from the anti-estrogen groups consisting of tamoxifen, raloxifene and toremifene, and from the hormonal groups consisting of progesterones and corticosteroids.

4. A method according to claim 1 wherein said prostatic implants of said drug compositions are made as separate or in combination thereof.

5. The method of claim 1 wherein said breast implants are made as biodegradable fused combinations of said therapeutic drug compositions and a lipoid carrier and said fused implants containing a single or multiples of said drug formulations for their slow release direct to breast.

6. A method according to claim 1 wherein said breast implants are made of Silastic capsules containing said therapeutic drug compositions as separate or in combination thereof for said formulation's slow release direct to breast.

7. The method of claim 1 wherein said breast implants are made as injectable microcapsules prepared from biodegradable polymer and said microcapsules containing said therapeutic drug compositions as separate or as in combination thereof for injection to the breast as slow release implant.

8. The method of claim 7, wherein said prostatic implants are made as injectable microcapsules prepared from biodegradable polymer and said microcapsules containing said therapeutic drug compositions dispensed in sterile liquid medium in sterile syringe for direct prostatic injection as slow release implant.

9. The method of claim 7, wherein said breast implants are made as injectable microcapsules prepared from biodegradable polymer and said microcapsules containing said therapeutic drug compositions dispensed in a mixture of sterile liquid mediums like normal saline, a local anesthetic and ethanol in a sterile syringe for direct injection to the breast as chelating slow release formulations when it comes in contact with breast tissue.

10. The method of claim 1 wherein said breast implants are selected from readily available commercial pharmaceutical preparations of anti-estrogens steroid hormones or their derivatives and said implants containing a single or multiples of said drug formulations for their slow release direct to the breast.

11. An improved method of concomitant hormonal and radiation treatment of the breast cancer and said hormonal treatment comprising of breast implants of anti-estrogens and steroid hormones in one or more slow release formulations and permitting said drugs to be continuously released at near constant rate directly to the breast during the radiation therapy and afterwards for longer periods.

12. An improved method of concomitant hormonal and radiation treatment of the breast cancer according to claim 11, wherein said continued slow release of hormonal composition directly to the breast during the interstitial radioactive seeds implants and afterwards for longer periods.

13. An improved method of concomitant hormonal and radiation treatment of breast cancer according to claim 11, wherein said hormonal implants to the breast is performed concomitantly with the radioactive implants to improve cure and convenience to patient than when they are implanted separately.

14. An improved method of treating hormone dependent and hormone refractory breast cancer and its accessible metastasis by implanting combinations of hormones and anti-estrogens to said tumor sites for improved tumor control with lesser toxicity than by administering said hormonal compositions at higher doses by mouth, subcutaneous, intramuscular or intravenous routes and said hormone compositions containing in one or more slow release implant formulations.

15. A method of claim 14, wherein said breast, subcutaneous or intramuscular hormonal implants methods comprising implanting single or synergetic combination of hormonally and cytotoxically effective compositions selected from the anti-estrogen groups consisting of tamoxifen, raloxifene and toremifene and from the hormonal groups consisting of iodo-estradiol, progesterones, corticosteroids and they are fused with a lipoid carrier or encapsulated in Silastic capsules or formulated as injectable microcapsules as suitable slow-release breast, subcutaneous or intramuscular implant.

16. A method of claim 14, wherein said slow-release breast implant of anti-estrogen and hormonal compositions for treating breast cancer and providing minimum or no toxicity as compared to when said drug compositions are administered by oral routs daily.

17. A method of claim 14, wherein high concentrations of said drug composition to the breast is achieved by implanting said formulations directly to the breast and to derive the added beneficial effect from these breast implants on breast cancer by inhibiting the hypothalamic-pituitary LHRH, FSH and LH secretion by these composition's systemic contents.

18. Slow-release anti-cancer breast implants products for treatment of breast cancer and comprising of anti-estrogens and steroid hormones as fused with a lipoid carrier or as encapsulated in Silastic capsules or as injectable microcapsules and are suitable for breast implantation such that said hormonally and cytotoxically effective compositions are continuously released at relatively high constant rates to the breast.

19. The said products of claim 18 being further characterized by providing effective tumor control and having minimum or no systemic toxicity associated with said composition's breast implants than if they were daily administered orally for several years at much higher doses to achieve the same results.

20. Slow-release anti-cancer prostate implant product of claim 18, wherein said single drug formulation is made from any one of the anti-estrogen drugs from a group consisting of tamoxifen, raloxifene or toremifene and hormonal compositions consisting of progesterone, androgens or prednisolone.

21. Slow-release anti-cancer breast implant product of claim 18, wherein said synergetic two drugs formulations comprises of an anti-estrogen from the groups of tamoxifen, raloxifene, or toremifene and a progesterone composition selected from the group consisting of megestrol acetate, medroxyprogesterone, norethindrone acetate or norgestrel or from the corticosteroids groups, the prednisolone.

22. Slow-release anti-cancer breast implant product of claim 18, wherein said synergetic three drugs formulations comprises of an anti-estrogen from the groups of tamoxifen, raloxifene, or toremifene and a progesterone composition selected from the group consisting of megestrol acetate, medroxyprogesterone, norethindrone acetate norgestrel and from the corticosteroids group prednisolone.

23. Anti-cancer products of claim 18, wherein said compositions comprising of single or synergetic combination of hormonally and cytotoxically effective amount of an anti-estrogen from the groups of tamoxifen, raloxifene, or toremifene and a progesterone composition selected from the group consisting of megestrol acetate, medroxyprogesterone, norethindrone acetate norgestrel and from the corticosteroids group prednisolone as fused with a lipoid carrier suitable for prostatic implantation.

24. Anticancer products according to claim 18, wherein said single or synergetic combination of hormonally and cytotoxically effective amounts of formulations as fused with a lipoid carrier suitable for breast implantation such that said compositions are continuously released at relatively constant rates to the breast for longer periods and the contents of said compositions being kept in amounts effective to suppress tumor growth with minimum or no systemic toxicity than if said drug compositions were administered daily by oral routes at much higher doses to achieve the same results as by said low dose breast implants.

25. Anticancer products of claim 18, wherein said single or synergetic combinations of hormonally and cytotoxically effective amounts of an anti-estrogen from the groups of tamoxifen, raloxifene, or toremifene and a progesterone composition selected from the group consisting of megestrol acetate, medroxyprogesterone, norethindrone acetate norgestrel and from the corticosteroids group prednisolone in same or separate slow release Silastic capsules suitable for prostatic implantation.

26. Anticancer products according to claim 18, wherein said single or synergetic combination of hormonally and cytotoxically effective formulations as in slow release Silastic capsules suitable for breast implantation such that said compositions are continuously released at relatively constant rates for longer periods and with minimum or no systemic toxicity than if said drug compositions were frequently administered orally at much higher doses to achieve the same results as by said low dose breast implants.

27. Anticancer products according to claim 18, wherein said anti-cancer products comprising of single or synergetic combination of hormonally and cytotoxically effective amounts of an anti-estrogen from the groups of tamoxifen, raloxifene, or toremifene and a progesterone composition selected from the group consisting of megestrol acetate, medroxyprogesterone, norethindrone acetate norgestrel and from the corticosteroids group prednisolone in same or separate slow release injectable microcapsules suitable for breast implantation.

28. Anticancer products according to claim 27, wherein said single or synergetic combination of hormonally and cytotoxically effective amount of formulations as injectable microcapsules suitable for breast implantation such that said compositions are continuously released at relatively constant rates and the contents of said compositions being kept in amounts effective to suppress tumor growth with minimum or no systemic toxicity than if said drug compositions were frequently administered orally at much higher doses to achieve the same results as by said low dose breast implants.

29. Anticancer products according to claim 18, wherein said implant products comprising of an anti-estrogen from the groups of tamoxifen, raloxifene, or toremifene and a progesterone composition selected from the group consisting of megestrol acetate, medroxyprogesterone, norethindrone acetate norgestrel and from the corticosteroids group prednisolone and are made as separate or as mixtures of two or more thereof and fused with a lipoid carrier.

30. Anti-cancer prostatic implant products according to claim 18, wherein said biodegradable breast implants comprising of an anti-estrogen from the groups of tamoxifen, raloxifene, or toremifene and a progesterone composition selected from the group consisting of megestrol acetate, medroxyprogesterone, norethindrone acetate norgestrel and from the corticosteroids group prednisolone and are made as separate or as mixtures of two or more thereof as injectable microcapsules.

31. Anti-cancer prostatic implant products of claim 18, wherein said breast implants comprises of an anti-estrogen from the groups of tamoxifen, raloxifene, or toremifene and a progesterone composition selected from the group consisting of megestrol acetate, medroxyprogesterone, norethindrone acetate norgestrel and from the corticosteroids group prednisolone and are made as separate or as mixtures of two or more thereof in Silastic capsules.

32. A slow-release hormonal breast implant method and products comprising single or synergetic combination of hormonally and cytotoxically effective compositions selected from the an anti-estrogen groups of tamoxifen, raloxifene, or toremifene and a progesterone composition selected from the group consisting of megestrol acetate, medroxyprogesterone, norethindrone acetate norgestrel and from the corticosteroids group prednisolone and they are fused with a lipoid carrier or encapsulated in Silastic capsules or formulated as injectable microcapsules as suitable slow-release breast implantation and implanting said products for the treatment of early and advanced stage breast cancers and as hormonal treatment combined with radiation.

33. A method and product of claim 32, wherein said hormone implant treatment of breast cancer is less-costly, less toxic and more convenient to the patient.

34. A method and product of claim 32, wherein said slow-release anti-estrogen implant treatment of the breast as hormonal prophylaxis against developing breast cancer by saturation of the breast tissue's binding sites for said anti-estrogens with high efficiency than by said anti-estrogen's daily oral administration at higher doses.
